(19) United States
(12) Patent Application Publication
(10) Pub. No.: US 2017/0319803 A1
(43) Pub. Date: Nov. 9, 2017

(54) DOUBLE CUFF TRACHEOSTOMY DEVICE

(71) Applicant: Mayo Foundation for Medical Education and Research, Rochester, MN (US)

(72) Inventors: Johnathon M. Aho, Rochester, MN (US); Cornelius A. Thiels, Rochester, MN (US); Blake A. Spindler, Rochester, MN (US); Alisha A. Lucas, Rochester, MN (US)

(73) Assignee: Mayo Foundation for Medical Education and Research, Rochester, MN (US)

(21) Appl. No.: 15/516,157

(22) PCT Filed: Sep. 29, 2015
(86) PCT No.: PCT/US15/52930

§ 371 Date: Mar. 31, 2017

Related U.S. Application Data
(60) Provisional application No. 62/059,492, filed on Oct. 3, 2014.

(51) Int. Cl. A61M 16/04

(52) U.S. Cl. CPC A61M 16/0434; A61M 16/0465; A61M 16/0402; A61M 16/044; A61M 16/0468

(57) ABSTRACT

This document provides devices and methods for performing tracheostomies. For example, this document provides devices and methods for performing tracheostomies using tracheostomy tubes that include a cuff at the neck opening area that can prevent or reduce the potential for air leaks.

### CROSS REFERENCE TO RELATED APPLICATIONS

This application claims the benefit of U.S. Provisional Ser. No. 62/059,492 filed Oct. 3, 2014. This disclosure of the prior application is considered part of (and is incorporated by reference in) the disclosure of this application.

### BACKGROUND

### 1. Technical Field

This document relates to devices and methods for performing tracheostomies. For example, this document relates to devices and methods for performing tracheostomies using tracheostomy tube devices that include a cuff at the neck opening area that can prevent or reduce the potential for air leaks.

### 2. Background Information

Airway access for mechanical ventilation can be provided either by endotracheal or tracheostomy tube. In the acute setting an endotracheal tube is typically used, but tracheostomy tubes are often used for people who will require long-term use of a ventilator. A tracheotomy is a surgically created hole through the front of the neck and into the trachea. A tracheotomy procedure is a generally safe and commonly done procedure.

Patients with tracheostomies are at risk of developing air leaks. In addition about 1% of patients with tracheostomies develop subcutaneous emphysema which can also become problematic. These are initially often managed by keeping the tracheostomy balloon inflated. However, this hinders pulmonary rehabilitation and prevents the patient from being able to speak. The other option requires upsizing the size of the tracheostomy tube which is not ideal and can result in not only a larger tracheotomy hole but also a risk of skin necrosis. Lastly, in some scenarios air leaks can be severe enough to result in respiratory decline and become an emergency.

### SUMMARY

This document provides devices and methods for performing tracheostomies. For example, this document provides devices and methods for performing tracheostomies using tracheostomy tube devices that include a cuff at the neck opening area that can prevent or reduce the potential for air leaks.

In one implementation, a tracheostomy tube device for treating a patient includes: a proximal tube portion configured to be positioned at least partially exterior to the patient when the device is installed in the patient; a tube extending from the proximal tube portion and configured to be positioned at least partially within a tracheostomy stoma and at least partially within a trachea of the patient when the device is installed in the patient; a tracheal cuff positioned on an exterior of the tube, the tracheal cuff being inflatable and positioned to contact an inner wall of the trachea when the device is installed in the patient; and a neck cuff positioned on the exterior of the tube, the neck cuff being inflatable and positioned to contact an inner wall of the tracheostomy stoma when the device is installed in the patient.

Such a tracheostomy tube device may optionally include one or more of the following features. The device may further comprise a neck flange positioned between the proximal tube portion and the tube. The neck flange may be configured to be positioned at least partially exterior to the patient when the device is installed in the patient. The device may further comprise a shared inflation device, wherein activation of the shared inflation device causes inflation of both the tracheal cuff and the neck cuff. The device may further comprise a shared inflation device and a valve, wherein activation of the shared inflation device when the valve is in a first arrangement causes inflation of the tracheal cuff but not the neck cuff, and wherein activation of the shared inflation device when the valve is in a second arrangement causes inflation of the neck cuff but not the tracheal cuff. The device may further comprise a first inflation device and a second inflation device, wherein the tracheal cuff is inflated by activation of the first inflation device and the neck cuff is inflated by activation of the second inflation device. The neck cuff may include a bulbous proximal end portion or a bulbous distal end portion. The neck cuff may include a bulbous proximal end portion and a bulbous distal end portion.

In another implementation, a tracheostomy tube device for treating a patient includes: a proximal tube portion configured to be positioned at least partially exterior to the patient when the device is installed in the patient; a tube extending from the proximal tube portion and configured to be positioned at least partially within a tracheostomy stoma and at least partially within a trachea of the patient when the device is installed in the patient; a tracheal cuff positioned on an exterior of the tube, the tracheal cuff being inflatable and positioned to contact an inner wall of the trachea when the device is installed in the patient; a proximal neck cuff positioned on the exterior of the tube, the proximal neck cuff being inflatable and positioned to contact a neck of the patient when the device is installed in the patient; and a distal neck cuff positioned on the exterior of the tube, the distal neck cuff being inflatable and positioned to contact the inner wall of the trachea when the device is installed in the patient.

Such a tracheostomy tube device may optionally include one or more of the following features. The device may further comprise a neck flange positioned between the proximal tube portion and the tube. The neck flange may be configured to be positioned at least partially exterior to the patient when the device is installed in the patient. The device may further comprise a shared inflation device, wherein activation of the shared inflation device causes inflation of each of the tracheal cuff, the proximal neck cuff, and the distal neck cuff. The device may further comprise a shared inflation device and a valve, wherein activation of the shared inflation device when the valve is in a first arrangement causes inflation of the tracheal cuff but not the proximal neck cuff and not the distal neck cuff, and wherein activation of the shared inflation device when the valve is in a second arrangement causes inflation of the proximal neck cuff and the distal neck cuff but not the tracheal cuff. The device may further comprise a first inflation device and a second inflation device, wherein the tracheal cuff is inflated by activation of the first inflation device and both the proximal neck cuff and the distal neck cuff are inflated by activation of the second inflation device.

In another implementation, a method for treating a patient using a tracheostomy tube device includes: creating a tracheostomy stoma in a neck of the patient; installing the tracheostomy tube device within the tracheostomy stoma and a trachea of the patient; and inflating the neck cuff to contact the inner wall of the tracheostomy stoma. The tracheostomy tube device comprises: a proximal tube portion positioned at least partially exterior to the patient; a tube extending from the proximal tube portion and positioned at least partially within the tracheostomy stoma and at least partially within the trachea of the patient; a tracheal cuff positioned on an exterior of the tube, the tracheal cuff being inflatable and positioned to contact an inner wall of the trachea; and a neck cuff positioned on the exterior of the tube, the neck cuff being inflatable and positioned to contact an inner wall of the tracheostomy stoma.

In some implementations of the method for treating a patient using a tracheostomy tube device, inflating the neck cuff at least partially seals an opening between the tracheostomy tube device and the inner wall of the tracheostomy stoma.

Particular embodiments of the subject matter described in this document can be implemented to realize one or more of the following advantages. In some implementations, by adding a second cuff to the tracheostomy tube device in the area of the neck opening, air leaks can be prevented and/or treated effectively without the need for up-sizing or keeping the inner cuff inflated. The second cuff can be inflated around the skin hole, and thus prevent or reduce the potential for an air leak around the skin hole. The second cuff can be inflated and deflated to varying degrees depending on the degree of air leak. Using a second cuff instead of a larger tracheotomy tube may prevent the neck hole from getting permanently larger, and may also result in less risk of skin necrosis. In addition, since the skin, subcutaneous tissue, and tracheal cartilage is not a smooth and round shape like the plastic tube of a tracheostomy tube, a second cuff will conform better and thus decrease the risk of air leak. Further, the additional second cuff may also help secure the tracheotomy tube in place, and decrease the risk of it accidentally falling out.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic illustration of a patient (in cross-section) that is being treated with a typical tracheostomy tube device.

FIG. 2 is another schematic illustration of a patient (in cross-section) that is being treated with a typical tracheostomy tube device.

FIG. 3 is another schematic illustration of a patient (in cross-section) that is being treated with an example tracheostomy tube device in accordance with some embodiments provided herein.

FIG. 4 is a side view of the tracheostomy tube device of FIG. 3.

FIG. 5 is another schematic illustration of a patient (in cross-section) that is being treated with another example tracheostomy tube device in accordance with some embodiments provided herein.

FIG. 6 is another schematic illustration of a patient (in cross-section) that is being treated with another example tracheostomy tube device in accordance with some embodiments provided herein.

Like reference numbers represent corresponding parts throughout.

### DETAILED DESCRIPTION

This document provides devices and methods for performing tracheostomies. For example, this document provides devices and methods for performing tracheostomies using tracheostomy tube devices that include a cuff at the neck opening area that can prevent or reduce the potential for air leaks.

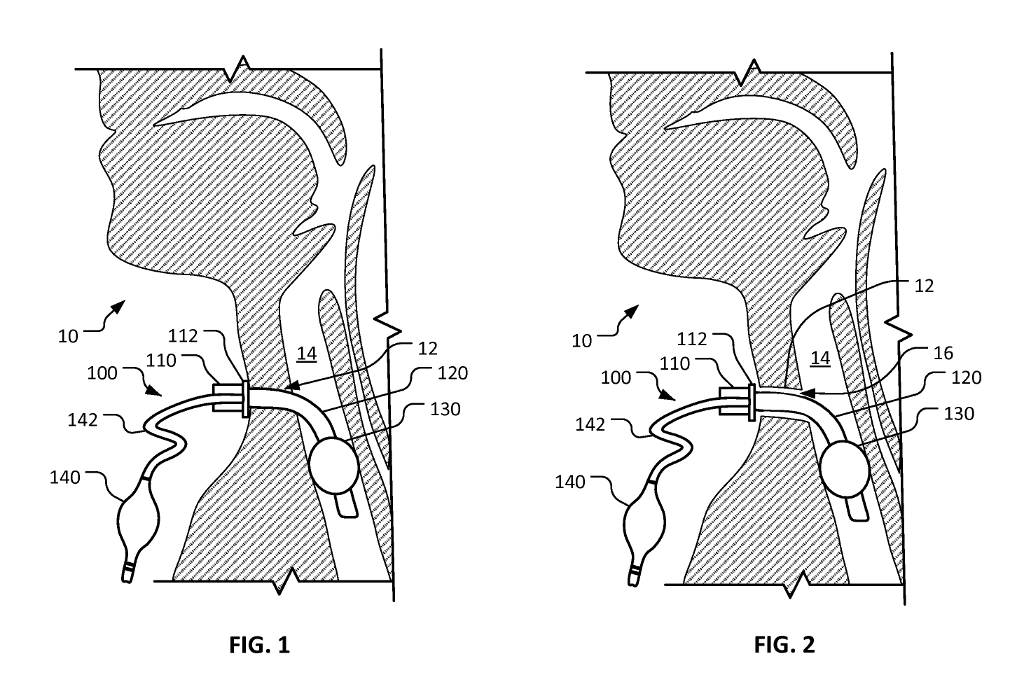

With reference to FIG. 1, a human patient 10 is being treated with a typical tracheostomy tube device 100. Patient 10 has a tracheostomy stoma 12 that provides surgical access to the patient's trachea 14 (airway).

Tracheostomy tube device 100 includes a proximal tube portion 110, a neck flange 112, a tube 120, a tracheal cuff 130, a pilot balloon 140, and a cuff inflation line 142. Proximal tube portion 110 is coupled to neck flange 112, and both are positioned external to patient 10. In some implementations, proximal tube portion 110 is a connector that can be used to attach a mechanical ventilator (not shown) to tracheostomy tube device 100. Tube 120 extends distally from neck flange 112, so as to pass through tracheostomy stoma 12 and into trachea 14. Tracheal cuff 130 is mounted on and surrounds tube 120. Tracheal cuff 130 is a balloon that can be inflated to provide a seal between tube 120 and an inner wall of trachea 14. Pilot balloon 140 and cuff inflation line 142 facilitate inflation of tracheal cuff 130.

With reference to FIG. 2, patient 10 is being treated with tracheostomy tube device 100. In this example, tracheostomy stoma 12 has enlarged beyond the outer size of tube 120. Therefore, a gap 16 exists between tracheostomy stoma 12 and tube 120. Gap 16 is clinically undesirable, and can result in various negative consequences to patient 10 as described above.

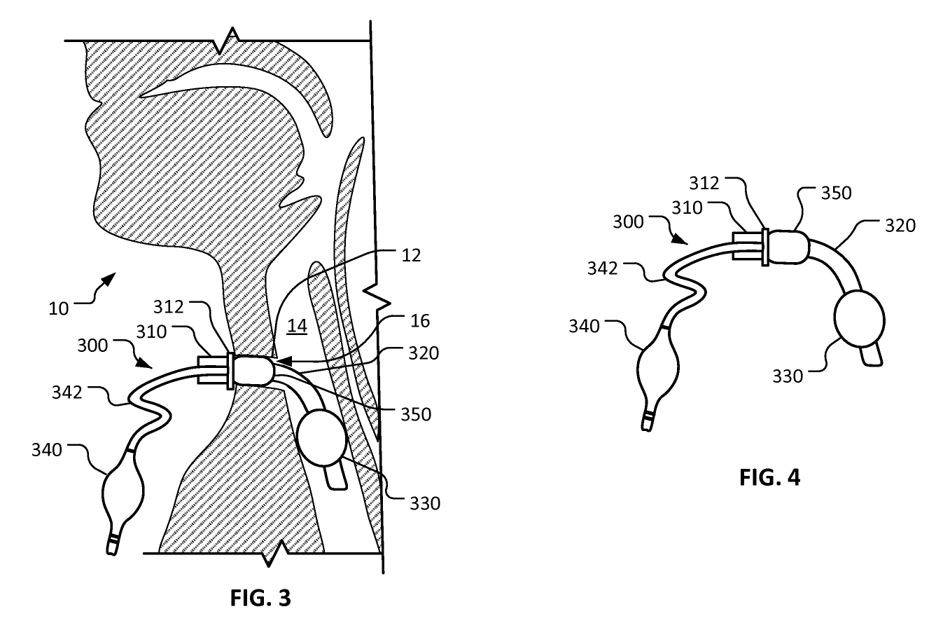

With reference to FIGS. 3 and 4, patient 10 is being treated with an example double cuff tracheostomy device 300. Double cuff tracheostomy device 300 includes a proximal tube portion 310, a neck flange 312, a tube 320, a tracheal cuff 330, a pilot balloon 340, a cuff inflation line 342, and a neck cuff 350. Proximal tube portion 310 is coupled to neck flange 312, and both are positioned external to patient 10. Tube 320 extends distally from neck flange 312, so as to pass through tracheostomy stoma 12 and into trachea 14. Tracheal cuff 330 is mounted on and surrounds tube 320. Pilot balloon 340 and cuff inflation line 342 facilitate inflation of tracheal cuff 330.

Neck cuff 350 at least partially surrounds a proximal end portion of tube 320. In some embodiments, neck cuff 350 is attached to neck flange 312. In some embodiments, neck cuff 350 is separated from neck flange 312. Neck cuff 350 can be inflated to fill a space that may exist between a proximal end portion of tube 320 and an inner wall of tracheostomy stoma 12 (such as gap 16 of FIG. 2, for example). In that manner, neck cuff 350 can be inflated to eliminate or reduce undesirable para-tracheostomy device air leaks through tracheostomy stoma 12.

In some embodiments, pilot balloon 340 and cuff inflation line 342 facilitate inflation of neck cuff 350, in addition to tracheal cuff 330. For example, in some embodiments both neck cuff 350 and tracheal cuff 330 are in fluid communication with pilot balloon 340 and cuff inflation line 342. Therefore, both neck cuff 350 and tracheal cuff 330 can be inflated by activating pilot balloon 340. In some such embodiments, a valve (not shown) is included in-line with cuff inflation line 342 by which pilot balloon 340 can be used to selectively inflate neck cuff 350 or tracheal cuff 330 individually.

In some embodiments, a second pilot balloon (not shown) to inflate neck cuff 350 can be included as a component part of double cuff tracheostomy device 300. In some embodiments, other inflation components or devices can be included in double cuff tracheostomy device 300 to inflate neck cuff 350. For example, in some embodiments a luer fitting with a one-way valve is provided for inflating one or both of neck cuff 350 or tracheal cuff 330. In such a case, a syringe can be used to inflate neck cuff 350 or tracheal cuff 330, for example.

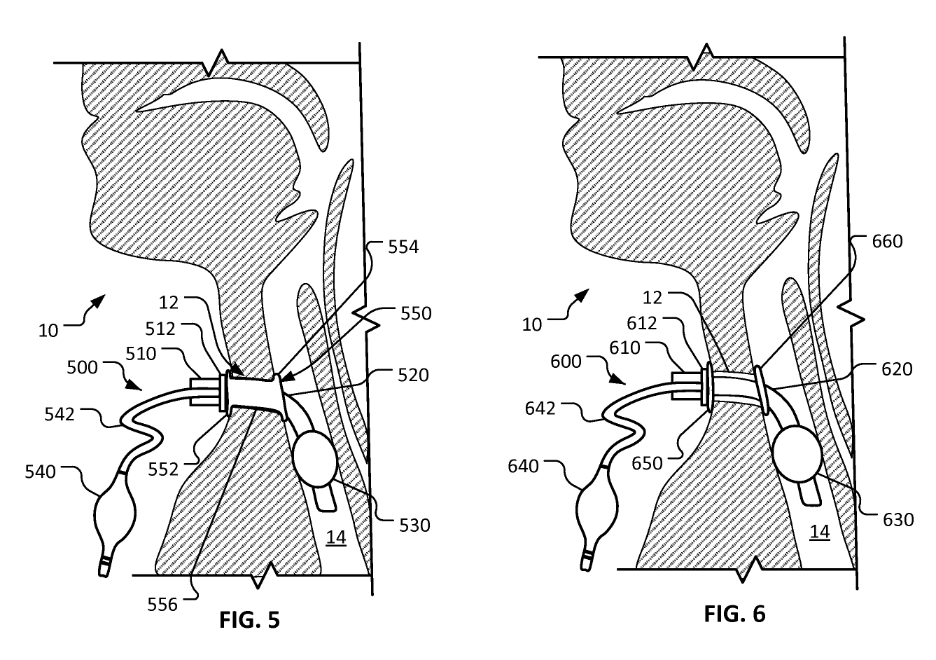

With reference to FIG. 5, patient 10 is being treated with another example double cuff tracheostomy device 500. Double cuff tracheostomy device 500 includes a proximal tube portion 510, a neck flange 512, a tube 520, a tracheal cuff 530, a pilot balloon 540, a cuff inflation line 542, and a neck cuff 550. Proximal tube portion 510 is coupled to neck flange 512, and both are positioned external to patient 10. Tube 520 extends distally from neck flange 512, so as to pass through tracheostomy stoma 12 and into trachea 14. Tracheal cuff 530 is mounted on and surrounds tube 520. Pilot balloon 540 and cuff inflation line 542 facilitate inflation of tracheal cuff 530.

Neck cuff 550 at least partially surrounds a proximal end portion of tube 520. In some embodiments, neck cuff 550 is attached to neck flange 512. In some embodiments, neck cuff 550 is separated from neck flange 512. Neck cuff 550 can be inflated to fill a space that may exist between a proximal end portion of tube 520 and tracheostomy stoma 12 (such as gap 16 of FIG. 2, for example). In that manner, neck cuff 550 can be inflated to eliminate or reduce undesirable para-tracheostomy device air leaks through tracheostomy stoma 12. Neck cuff 550 can be inflated in any of the manners described above in reference to neck cuff 350.

In the depicted embodiment, neck cuff 550 includes a bulbous proximal end portion 552, a bulbous distal end portion 554, and a middle portion 556. In some embodiments, neck cuff 550 includes a single bulbous end portion, such as either proximal end portion 552 or bulbous distal end portion 554. In some implementations, bulbous proximal end portion 552 can be inflated to seal tracheostomy stoma 12 at the exterior opening of tracheostomy stoma 12, such that bulbous proximal end portion 552 is at least partially exterior to patient 10. In some implementations, bulbous distal end portion 554 can be inflated to seal tracheostomy stoma 12 at the interior opening of tracheostomy stoma 12, such that bulbous distal end portion 554 is at least partially within trachea 14.

With reference to FIG. 6, patient 10 is being treated with an example triple cuff tracheostomy device 600. Triple cuff tracheostomy device 600 includes a proximal tube portion 610, a neck flange 612, a tube 620, a tracheal cuff 630, a pilot balloon 640, a cuff inflation line 642, a proximal neck cuff 650, and a distal neck cuff 660. Proximal tube portion 610 is coupled to neck flange 612, and both are positioned external to patient 10. Tube 620 extends distally from neck flange 612, so as to pass through tracheostomy stoma 12 and into trachea 14. Tracheal cuff 630 is mounted on and surrounds tube 620. Pilot balloon 640 and cuff inflation line 642 facilitate inflation of tracheal cuff 630.

Neck cuffs 650 and 660 at least partially surround a proximal end portion of tube 620. In some embodiments, proximal neck cuff 650 is attached to neck flange 612. In some embodiments, proximal neck cuff 650 is separated from neck flange 612. Neck cuffs 650 and 660 can be inflated to seal a space that may exist between a proximal end portion of tube 620 and tracheostomy stoma 12 (such as gap 16 of FIG. 2, for example). In that manner, neck cuffs 650 and 660 can be inflated to eliminate or reduce undesirable para-tracheostomy device air leaks through tracheostomy stoma 12. Neck cuffs 650 and 660 can be inflated in any of the manners described above in reference to neck cuff 350. In some embodiments, neck cuffs 650 and 660 are inflated individually. In some embodiments, neck cuffs 650 and 660 are inflated in combination with each other.

In some implementations, proximal neck cuff 650 can be inflated to seal tracheostomy stoma 12 at the exterior opening of tracheostomy stoma 12, such that proximal neck cuff 650 is at least partially exterior to patient 10. In some implementations, distal neck cuff 660 can be inflated to seal tracheostomy stoma 12 at the interior opening of tracheostomy stoma 12, such that distal neck cuff 660 is at least partially within trachea 14.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A tracheostomy tube device for treating a patient, the device comprising:
a proximal tube portion configured to be positioned at least partially exterior to the patient when the device is installed in the patient;
a tube extending from the proximal tube portion and configured to be positioned at least partially within a tracheostomy stoma and at least partially within a trachea of the patient when the device is installed in the patient;
a tracheal cuff positioned on an exterior of the tube, the tracheal cuff being inflatable and positioned to contact an inner wall of the trachea when the device is installed in the patient; and
a neck cuff positioned on the exterior of the tube, the neck cuff being inflatable and positioned to contact an inner wall of the tracheostomy stoma when the device is installed in the patient.

2. The device of claim 1, further comprising a neck flange positioned between the proximal tube portion and the tube, the neck flange configured to be positioned at least partially exterior to the patient when the device is installed in the patient.

3. The device of claim 1, further comprising a shared inflation device, wherein activation of the shared inflation device causes inflation of both the tracheal cuff and the neck cuff.

4. The device of claim 1, further comprising a shared inflation device and a valve, wherein activation of the shared inflation device when the valve is in a first arrangement causes inflation of the tracheal cuff but not the neck cuff, and wherein activation of the shared inflation device when the valve is in a second arrangement causes inflation of the neck cuff but not the tracheal cuff.

5. The device of claim 1, further comprising a first inflation device and a second inflation device, wherein the tracheal cuff is inflated by activation of the first inflation device and the neck cuff is inflated by activation of the second inflation device.

6. The device of claim 1, wherein the neck cuff includes a bulbous proximal end portion or a bulbous distal end portion.

7. The device of claim 1, wherein the neck cuff includes a bulbous proximal end portion and a bulbous distal end portion.

8. A tracheostomy tube device for treating a patient, the device comprising:
a proximal tube portion configured to be positioned at least partially exterior to the patient when the device is installed in the patient;
a tube extending from the proximal tube portion and configured to be positioned at least partially within a tracheostomy stoma and at least partially within a trachea of the patient when the device is installed in the patient;
a tracheal cuff positioned on an exterior of the tube, the tracheal cuff being inflatable and positioned to contact an inner wall of the trachea when the device is installed in the patient;
a proximal neck cuff positioned on the exterior of the tube, the proximal neck cuff being inflatable and positioned to contact a neck of the patient when the device is installed in the patient; and
a distal neck cuff positioned on the exterior of the tube, the distal neck cuff being inflatable and positioned to contact the inner wall of the trachea when the device is installed in the patient.

9. The device of claim 8, further comprising a neck flange positioned between the proximal tube portion and the tube, the neck flange configured to be positioned at least partially exterior to the patient when the device is installed in the patient.

10. The device of claim 8, further comprising a shared inflation device, wherein activation of the shared inflation device causes inflation of each of the tracheal cuff, the proximal neck cuff, and the distal neck cuff.

11. The device of claim 8, further comprising a shared inflation device and a valve, wherein activation of the shared inflation device when the valve is in a first arrangement causes inflation of the tracheal cuff but not the proximal neck cuff and not the distal neck cuff, and wherein activation of the shared inflation device when the valve is in a second arrangement causes inflation of the proximal neck cuff and the distal neck cuff but not the tracheal cuff.

12. The device of claim 8, further comprising a first inflation device and a second inflation device, wherein the tracheal cuff is inflated by activation of the first inflation device and both the proximal neck cuff and the distal neck cuff are inflated by activation of the second inflation device.

13. A method for treating a patient using a tracheostomy tube device, the method comprising:
creating a tracheostomy stoma in a neck of the patient;
installing the tracheostomy tube device within the tracheostomy stoma and a trachea of the patient, wherein the tracheostomy tube device comprises:
a proximal tube portion positioned at least partially exterior to the patient;
a tube extending from the proximal tube portion and positioned at least partially within the tracheostomy stoma and at least partially within the trachea of the patient;
a tracheal cuff positioned on an exterior of the tube, the tracheal cuff being inflatable and positioned to contact an inner wall of the trachea; and
a neck cuff positioned on the exterior of the tube, the neck cuff being inflatable and positioned to contact an inner wall of the tracheostomy stoma; and
inflating the neck cuff to contact the inner wall of the tracheostomy stoma.

14. The method of claim 13, wherein the inflating the neck cuff at least partially seals an opening between the tracheostomy tube device and the inner wall of the tracheostomy stoma.
